(19) United States
(12) Patent Application Publication
(10) Pub. No.: US 2026/0132116 A1
(43) Pub. Date: May 14, 2026

(54) CHRYSIN DERIVATIVE AND PHARMACEUTICAL COMPOSITION FOR TREATMENT OF INFLAMMATION AND TUBERCULOSIS COMPRISING THE SAME

(71) Applicants: KOREA ATOMIC ENERGY RESEARCH INSTITUTE, Daejeon (KR); KYUNGPOOK NATIONAL UNIVERSITY INDUSTRY-ACADEMIC COOPERATION FOUNDATION, Daegu (KR)

(72) Inventors: Ha Yeon SONG, Jeongeup-si (KR); Eui Baek BYUN, Daejeon (KR); Yu Na LEE, Paju-si (KR); Jong Ho JEON, Daegu (KR)

(73) Assignees: KOREA ATOMIC ENERGY RESEARCH INSTITUTE, Daejeon (KR); KYUNGPOOK NATIONAL UNIVERSITY INDUSTRY-ACADEMIC COOPERATION FOUNDATION, Daegu (KR)

(21) Appl. No.: 19/380,004

(22) Filed: Nov. 5, 2025

(30) Foreign Application Priority Data
Nov. 8, 2024 (KR) 10-2024-0157853
Oct. 23, 2025 (KR) 10-2025-0155078

(51) Int. Cl. C07D 311/30; A61K 31/352; A61P 29/00; A61P 31/06

(52) U.S. Cl. CPC C07D 311/30; A61K 31/352; A61P 29/00; A61P 31/06

(57) ABSTRACT

A chrysin derivative and a composition for a treatment of tuberculosis containing the same are provided. In detail, a chrysin derivative compound represented by chemical formula 1,

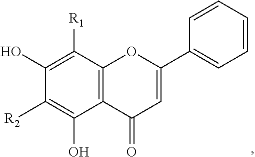

or a salt thereof, and a composition for a treatment of tuberculosis and inflammation containing the same as an active ingredient are provided. In chemical formula 1, at least one of R₁ and R₂ is hydrogen, and the other is a C₁₋₆ hydroxyalkyl group or a C₁₋₁₀ alkenyl group unsubstituted or substituted with one or more C₁₋₁₀ alkyl groups

### CROSS-REFERENCE TO RELATED APPLICATION(S)

This application claims benefit of priorities to Korean Patent Application No. 10-2024-0157853 filed on Nov. 8, 2024 and Korean Patent Application No. 10-2025-0155078 filed on Oct. 23, 2025 in the Korean Intellectual Property Office, the disclosure of which is incorporated herein by reference in its entirety.

### BACKGROUND

### 1. Field

The present disclosure relates to a chrysin derivative and a composition for treating inflammation and tuberculosis comprising the same, and more specifically, to providing a chrysin derivative having a novel structure and providing a pharmaceutical composition for treating inflammatory diseases and tuberculosis lesions using the same.

### 2. Description of Related Art

Chrysin, a flavonoid compound found primarily in propolis, honey and the like, is known to possess diverse pharmacological effects. Chrysin has been reported to exhibit various physiological activities, including anti-inflammatory, antioxidant, anticancer, antiviral, antibacterial, and neuroprotective effects. Due to these diverse pharmacological effects, chrysin is attracting attention as a substance with great potential for pharmaceutical development. For example, Korean Patent Application No. 10-2019-0163885 discloses an anti-inflammatory composition comprising CAPE, quercetin, and chrysin.

However, the use of chrysin presents several significant limitations. The most significant of these is its low bioavailability of less than approximately 1%. Chrysin is poorly absorbed when administered orally and is rapidly metabolized, making it difficult to maintain effective concentrations in the body. This poses a significant obstacle to developing chrysin as a pharmaceutical, and the high doses are required to fully achieve the pharmacological effects of chrysin, which may increase potential side effects.

The inflammatory response is a series of immune responses that inevitably occur due to activated immune cells. When immune cells are exposed to external substances, such as bacteria, viruses, and other microorganisms, or foreign substances, the immune cells become activated and accelerate the inflammatory response. When lipopolysaccharide (LPS), a bacterial inflammatory agent, triggers an inflammatory response, various pro-inflammatory mediators are produced. These pro-inflammatory mediators include nitric oxide (NO), produced by inducible nitric oxide synthase (iNOS), prostaglandin E₂ (PGE₂), produced by cyclooxygenase (COX-2), and the like.

Meanwhile, tuberculosis (TB) is an infectious disease caused by Mycobacterium tuberculosis. When airborne Mycobacterium tuberculosis are inhaled by people nearby, Mycobacterium tuberculosis enters the lungs and causes infection. When infected with Mycobacterium tuberculosis, macrophages, fibroblasts, and dendritic cells are activated, causing local inflammation and granulomas. Infection with Mycobacterium tuberculosis is a chronic inflammatory disease in which regulatory and proinflammatory processes are mutually or stepwise triggered, and may affect the development and progression of other diseases. Meanwhile, cytokines such as interferon, interleukin, and tumor necrosis factor, and cells such as macrophages, fibroblasts, regulatory T cells, and type 1 helper lymphocytes are key factors involved in tuberculosis inflammation. These factors have dual functions, either promoting or suppressing local inflammatory responses, and may be modulated by immune responses mediated by cytokines such as TNF-α, IL-6, IL-12, and IL-1β.

In patients with early-stage tuberculosis infection, the spread of Mycobacterium tuberculosis may be suppressed without complete eradication, leading to progression to chronic tuberculosis. As tuberculosis progresses to chronic tuberculosis, Mycobacterium tuberculosis form granulomas by phagocytes. These granulomas are surrounded by a peripheral mantle of fibroblasts. The primary function of fibroblasts has been reported to be to contain or isolate mycobacteria by secreting collagen at the periphery of the granuloma. Fibroblasts are the most abundant cell type within connective tissues susceptible to tuberculosis infection, supporting active tuberculosis replication. The interaction of fibroblasts with other immune cells is an integral component of the granuloma structures that develop in response to tuberculosis infection, leading to immunomodulatory functions that control tuberculosis infection.

Tuberculosis may be treated with long-term antibiotic therapy, but this treatment is insufficient to prevent the spread of tuberculosis and may lead to the development of drug resistance during antibiotic treatment, and thus there is a need for new effective treatments for tuberculosis.

### SUMMARY

An aspect of the present disclosure is to provide a novel chrysin derivative applicable to the treatment of tuberculosis.

An aspect of the present disclosure is to provide a composition comprising a novel chrysin derivative, effective for the treatment of tuberculosis.

An aspect of the present disclosure is to provide a composition comprising a novel chrysin derivative, effective for the treatment of inflammation.

According to an aspect of the present disclosure, a chrysin derivative compound represented by the following chemical formula 1 or a salt thereof is provided.

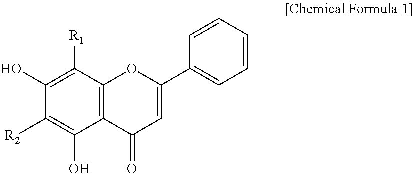

In chemical formula 1, at least one of R₁ and R₂ is hydrogen, and the other is a C₁₋₆ hydroxyalkyl group or a C₁₋₁₀ alkenyl group unsubstituted or substituted with one or more C₁₋₁₀ alkyl groups.

According to an aspect of the present disclosure, a composition for a treatment of tuberculosis, comprising a chrysin derivative compound represented by chemical formula 1, or a salt thereof as an active ingredient, is provided.

According to an aspect of the present disclosure, a composition for a treatment of inflammation, comprising a chrysin derivative compound represented by chemical formula 1, or a salt thereof as an active ingredient, is provided.

### BRIEF DESCRIPTION OF DRAWINGS

The above and other aspects, features, and advantages of the present disclosure will be more clearly understood from the following detailed description, taken in conjunction with the accompanying drawings, in which:

FIGS. 1A and 1B illustrate the results of the WST-1 assay for the cytotoxicity of each of the synthesized chrysin derivative compounds 1a, 1b, 5, 8, and 10, in the untreated state (FIG. 1A) and in the LPS (200 ng/mL)-treated state (FIG. 1B);

FIG. 2 illustrates the results of evaluating the potential of each synthesized chrysin derivative compound to inhibit IL-6 production in LPS-stimulated RAW264.7 cells;

FIG. 3A to FIG. 3D illustrates the results of evaluating the IL-6, TNF-α, PGE-2, and COX-2 inhibitory effects of each synthesized chrysin derivative in response to LPS stimulation;

FIGS. 4A and 4B illustrate the results of evaluating the IL-6 (FIG. 4A) and TNF-α (FIG. 4B) inhibitory effects of each synthesized chrysin derivative in response to stimulation of trehalose-6,6′-dimycolate (TDM), a glycolipid derived from Mycobacterium tuberculosis;

### DETAILED DESCRIPTION

Hereinafter, example embodiments will be described with reference to the attached drawings. However, the embodiments may be modified in various other forms, and the scope of the present disclosure is not limited to the embodiments described below.

In this specification, “Cx-Cy” indicates that the number of carbon atoms constituting the substituent is x to y. For example, “C₁-C₆” indicates that the number of carbon atoms constituting the substituent is 1 to 6.

According to an embodiment of the present disclosure, a novel chrysin derivative compound is provided, and the novel chrysin derivative compound according to an embodiment may be used in the treatment of tuberculosis.

More specifically, the novel chrysin derivative compound according to an embodiment is a chrysin derivative compound represented by the following chemical formula 1 or a salt thereof.

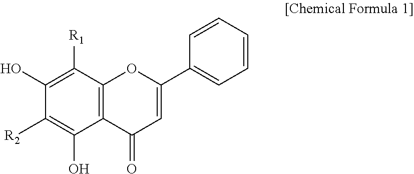

In chemical formula 1, at least one of R₁ and R₂ is hydrogen, and the other is a C₁₋₆ hydroxyalkyl group or a C₁₋₁₀ alkenyl group unsubstituted or substituted with one or more C₁₋₁₀ alkyl groups.

As used herein, the term “alkyl group” refers to a linear or branched saturated aliphatic hydrocarbon monovalent group. Specific examples include a methyl group, an ethyl group, a propyl group, an isobutyl group, a sec-butyl group, a tert-butyl group, a pentyl group, a hexyl group, and the like.

As used herein, the term “hydroxyalkyl group” refers to a monovalent group in which one or more —OH (hydroxyl groups) are substituted on any carbon of a linear or branched saturated aliphatic hydrocarbon. Specific examples include a hydroxymethyl group, a hydroxyethyl group, a hydroxypropyl group, and the like. The position of the carbon atom on which the hydroxyl group is substituted is not particularly limited.

As used herein, the term “alkenyl group” refers to a linear or branched unsaturated aliphatic hydrocarbon monovalent group containing one or more carbon-carbon double bonds, and may be, for example, an allyl group.

The salt of the chrysin derivative compound according to an embodiment may be in the form of a pharmaceutically acceptable salt. The term “pharmaceutically acceptable salt” as used herein refers to a formulation of the compound that does not cause serious irritation to the organism to which the compound is administered and does not impair the biological activity and physical properties of the compound. The pharmaceutical salts include acid addition salts formed by acids that form non-toxic acid addition salts containing pharmaceutically acceptable anions, for example, inorganic acids such as hydrochloric acid, sulfuric acid, nitric acid, phosphoric acid, hydrobromic acid (bromic acid), hydroiodic acid (iodic acid), perchloric acid and tartaric acid; organic carboxylic acids such as succinic acid, oxalic acid, mandelic acid, propionic acid, citric acid, lactic acid, glycolic acid, gluconic acid, tartaric acid, formic acid, citric acid, acetic acid, trichloroacetic acid, trifluoroacetic acid, gluconic acid, benzoic acid, lactic acid, fumaric acid, maleic acid, and salicylic acid; sulfonic acids such as methanesulfonic acid, ethanesulfonic acid, benzenesulfonic acid, p-toluenesulfonic acid, and naphthalenesulfonic acid; or the like. The compound according to an embodiment may be converted into a salt thereof by a method of the related art.

The compound of chemical formula (1) in the present disclosure may be synthesized by any synthetic process widely known in the art, and the synthetic steps are not limited thereto.

The compound of chemical formula (1) in the present disclosure may be provided, for example, in chemical formula 1 in which at least one of R₁ and R₂ is hydrogen, and the other is a C₁₋₁₀ allyl group unsubstituted or substituted with hydroxyethyl or one or two methyl groups. In this case, the allyl group substituted with one or two methyl groups may be, for example, a methylallyl group or a dimethylallyl group.

Preferably, the chrysin derivative compound represented by chemical formula 1 is at least one selected from the group consisting of Compound 1a, Compound 1b, Compound 5, and Compound 8.

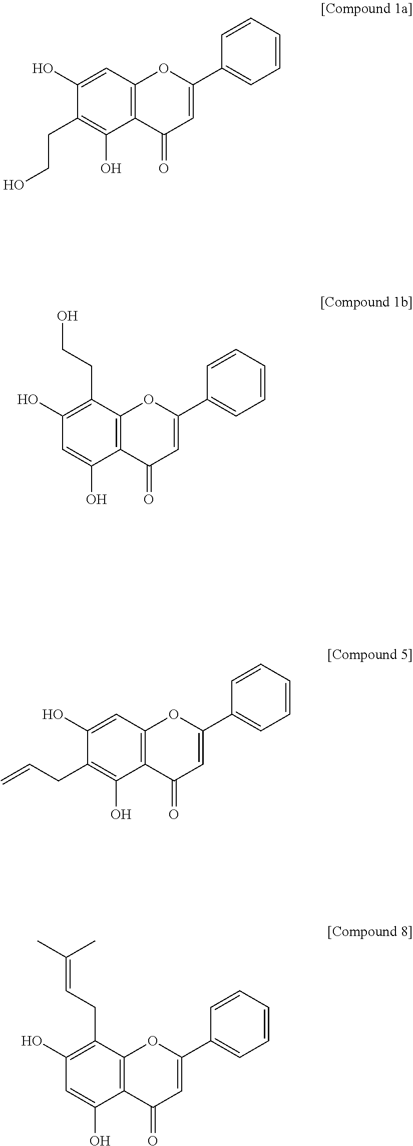 [SMILES: O=c1cc(-c2ccccc2)oc2cc(O)c(CCO)c(O)c12 ; O=c1cc(-c2ccccc2)oc2c(CCO)c(O)cc(O)c12 ; C=CCc1c(O)cc2oc(-c3ccccc3)cc(=O)c2c1O ; CC(C)=CCc1c(O)cc(O)c2c(=O)cc(-c3ccccc3)oc12]

According to another aspect of the present disclosure, a method for synthesizing the chrysin derivative compound according to an embodiment is provided.

The method for synthesizing the chrysin derivative compound according to an embodiment is not particularly limited, and known synthetic techniques may be applied without limitation. For example, the method for synthesizing the chrysin derivative compound according to an embodiment may include processes such as protection of the hydroxyl group using chrysin as a starting material, selective functionalization with an allyl or prenyl group, or the like, deprotection after rearrangement, and selective oxidation/reduction.

More specifically, the compound may be obtained by the following process. For example, to ensure regioselectivity, the 7-hydroxyl of chrysin is selectively protected with a methoxymethyl (MOM) group, leaving the 5-hydroxyl as the reactive site. Subsequently, nucleophilic substitution (O-alkylation) using allyl bromide, 3,3-dimethylallyl(prenyl) bromide, or the like in the presence of potassium carbonate is performed to produce the 5-O-allyl or 5-O-prenyl ether precursor respectively. The O-alkyl ether thus formed is heated in a high-boiling arylamine solvent to undergo a thermal Claisen rearrangement, converting the O-alkylated intermediate to an ortho-C-alkylated aryl skeleton. This step forms a new C—C bond, forming the C-alkylated intermediate. The MOM protecting group is then removed under acidic conditions to restore the free phenol, yielding compounds such as Compounds 5 and 8.

If necessary, after the reaction is complete, the product may be purified through related art liquid-liquid separation (EtOAc/aqueous phase), drying with anhydrous MgSO₄, concentration under reduced pressure, MPLC purification, or the like.

As another example, for Compounds 1a and 1b, the C-alkylated intermediate is used as a starting material, followed by dehydroxylation and glycol cleavage, to be converted to an aldehyde. Subsequently, reduction with NaBH₄ or the like may be performed to provide a primary alcohol, and finally, acidic deprotection may be performed to obtain compounds such as Compounds 1a and 1b.

According to another aspect of the present disclosure, a composition for the prevention and/or improvement and/or treatment of inflammation and inflammation-related diseases, and the prevention and/or improvement and/or treatment of tuberculosis, comprising the chrysin derivative compound according to an embodiment or a salt thereof as an active ingredient, is provided. Hereinafter, the respective terms “composition for a treatment of inflammation” and “composition for a treatment of tuberculosis” refer to pharmaceutical compositions that may also have preventive and improvement effects.

As used herein, the term “prevention” refers to any action that inhibits or delays the onset of a disease by administering the composition. In the present disclosure, “improvement” or “treatment” refers to any action that improves or beneficially alters the symptoms of the disease by administering the composition.

The composition for a treatment of inflammation may comprise the compounds of chemical formulae 1a and 8 of the present disclosure in an amount of 0.00001 wt % to 90 wt %, based on the total weight of the composition, for example, 0.001 to 50 wt %, preferably 0.01 to 10 wt %. If the amount of the compounds of chemical formulae 1a and 8 falls below the above range, the anti-inflammatory effect may be minimal, and if it exceeds the above range, toxicity may occur.

The composition for a treatment of tuberculosis comprises a chrysin derivative compound represented by the above-described chemical formula 1 or a salt thereof as an active ingredient.

At this time, the composition for a treatment of tuberculosis may contain the chemical formula 1 compound of the present disclosure or a salt thereof in an amount of 0.00001 wt % to 90 wt % based on the total weight of the composition, for example, 0.001 wt % to 50 wt %, preferably 0.01 wt % to 10 wt %. However, the effective amount is not particularly limited thereto.

The inflammatory disease to which the composition for a treatment of inflammation according to an embodiment of the present disclosure may be applied may be one of asthma, allergic and non-allergic rhinitis, chronic and acute rhinitis, chronic and acute gastritis, chronic and acute enteritis, ulcerative gastritis, acute and chronic nephritis, acute and chronic hepatitis, chronic obstructive pulmonary disease, pulmonary fibrosis, irritable bowel syndrome, inflammatory bowel disease, inflammatory pain, migraine, headache, back pain, fibromyalgia, myofascial disease, viral infection, bacterial infection, fungal infection, burns, surgical wounds, prostaglandin E hyperactivity syndrome, atherosclerosis, gout, arthritis, rheumatoid arthritis, ankylosing spondylitis, Hodgkin's disease, pancreatitis, conjunctivitis, iritis, scleritis, uveitis, dermatitis, atopic dermatitis, eczema, and multiple sclerosis, and includes, for example, inflammation caused by tuberculosis.

In the present disclosure, the tuberculosis may be derived from a Mycobacterium strain. For example, the Mycobacterium strain may be one or more strains selected from the group consisting of: Mycobacterium tuberculosis erdman, Mycobacterium tuberculosis, Mycobacterium bovis, Mycobacterium africanum, Mycobacterium microti, Mycobacterium fortuitum, Mycobacterium avium, Mycobacterium xenopi, Mycobacterium smegmatis, Mycobacterium tuberculosis H37Rv, Mycobacterium tuberculosis K, Mycobacterium kansasii, Mycobacterium marinum, Mycobacterium fortuitum, and Mycobacterium chelonae.

Meanwhile, in the present disclosure, the tuberculosis may be at least one of extrapulmonary tuberculosis and pulmonary tuberculosis in at least one area of the lymph nodes, gastrointestinal tract, joints, meninges, and genitourinary organs, and for example, may be at least one selected from the group consisting of ocular tuberculosis, skin tuberculosis, kidney tuberculosis, lymphatic tuberculosis, laryngeal tuberculosis, intestinal tuberculosis, pulmonary tuberculosis, biliary tuberculosis, bone tuberculosis, pharyngeal tuberculosis, breast tuberculosis, and spinal tuberculosis.

More specifically, the composition for a treatment of tuberculosis according to an embodiment may be for the treatment of inflammation caused by tuberculosis. For example, the inflammation caused by tuberculosis may be at least one selected from the group consisting of chronic inflammation and granulomas.

The chrysin derivative compound represented by Chemical Formula 1 or a salt thereof, included as an active ingredient in the composition for a treatment of tuberculosis according to an embodiment, may suppress an inflammatory mediator caused by Trehalose-6,6′-dimycolate (TDM). The inflammatory mediator may be at least one of TNF-αr and IL-6.

The pharmaceutical composition may be formulated into various forms, such as oral formulations such as powders, granules, tablets, capsules, suspensions, emulsions, syrups, and aerosols, and sterile injection solutions, according to methods of the related art, depending on the intended use. The pharmaceutical composition may be administered orally or through various routes, including intravenous, intraperitoneal, subcutaneous, intramuscular, intrathecal, rectal, and topical administration. Preferably, the pharmaceutical composition is formulated into an injectable preparation that may be administered by a route selected from the group consisting of intraperitoneal, subcutaneous, and intravenous, such as intravenous, intraperitoneal, subcutaneous, intramuscular, and intrathecal.

Examples of suitable carriers, excipients, and diluents that may be included in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinyl pyrrolidone, water, methyl hydroxybenzoate, propyl hydroxybenzoate, talc, magnesium stearate, mineral oil, and the like.

Furthermore, the pharmaceutical composition may further include fillers, anticoagulants, lubricants, wetting agents, flavoring agents, emulsifiers, preservatives, and the like.

Solid preparations for oral administration include tablets, pills, powders, granules, capsules, and the like. These solid preparations may be formulated by mixing the therapeutic pharmaceutical composition with at least one excipient, such as starch, calcium carbonate, sucrose, lactose, or gelatin. In addition to simple excipients, lubricants such as magnesium stearate and talc may also be used. Examples of liquid preparations for oral administration include suspensions, oral solutions, emulsions, syrups, and the like. In addition to commonly used simple diluents such as water and liquid paraffin, various excipients such as wetting agents, sweeteners, flavoring agents, and preservatives may be included.

Formulations for parenteral administration include sterile aqueous solutions, non-aqueous solvents, suspensions, emulsions, lyophilized preparations, and suppositories. Non-aqueous solvents and suspending agents include propylene glycol, polyethylene glycol, vegetable oils such as olive oil, and injectable esters such as ethyl oleate. The base of the injection may include additives of the related art such as solubilizers, isotonic agents, suspending agents, emulsifiers, stabilizers, and preservatives.

The composition according to an embodiment of the present disclosure as described above may also be administered in combination with known anti-tuberculosis agents. More specifically, the pharmaceutical composition according to an embodiment may be administered as an individual treatment or in combination with other treatments or treatment methods, and may be administered sequentially or simultaneously with treatments of the related art, or in single or multiple administrations. Considering all of the above factors, it is important to administer an amount that obtains maximum effect with the minimum amount possible without side effects, and this may be readily determined by those skilled in the art.

The appropriate dosage of the pharmaceutical composition according to an embodiment may vary depending on the patient's condition, weight, age, severity of the disease, drug form, route of administration, and duration of administration, and may be appropriately selected by those skilled in the art.

However, the dosage may vary depending on the route of administration, disease severity, gender, weight, age, or the like, and thus does not limit the scope of the present disclosure in any way.

According to another aspect of the present disclosure, a method for treating tuberculosis is provided, comprising administering the composition for a treatment of tuberculosis according to an embodiment to a patient with tuberculosis.

The composition for a treatment of tuberculosis according to an embodiment may be administered to a patient with tuberculosis in a pharmaceutically effective amount.

In the present disclosure, “administration” means providing a predetermined substance to a patient by any suitable method. The route of administration of the composition according to an embodiment of the present disclosure may be oral or parenteral through any common route as long as it may reach the target tissue, and oral administration may be preferred.

In the present disclosure, “patient” refers to a human or any other animal suffering from a disease whose symptoms may be improved by administering the composition according to an embodiment of the present disclosure. The composition according to an embodiment may be applied not only to humans (for treatment, suppression, or prevention) but also to other commercially useful animals, such as non-human animals.

In the present disclosure, a “pharmaceutically effective amount” refers to an amount sufficient to treat a disease with a reasonable benefit/risk ratio applicable to medical treatment. The level of the effective amount may be determined based on factors such as the type and severity of the patient's disease, drug activity, drug sensitivity, administration time, route and excretion rate, treatment duration, concurrent medications, and other factors well known in the medical field.

The composition for a treatment of tuberculosis according to an embodiment not only effectively suppresses general inflammatory responses, but may also be effectively applied as host-directed therapy, particularly to alleviate pulmonary granulomas in the treatment of tuberculosis.

The present disclosure will be described in more detail below through detailed examples. The following examples are provided merely as examples to aid understanding of the present disclosure and are not intended to limit the scope of the present disclosure.

### Example

### 1. Synthesis of Chrysin Derivatives

Derivatives 5, 8, 10, 1a, and 1b were obtained from chrysin according to the experimental method below. Previously reported HE-chrysin corresponds to 1b.

### 1) Synthesis of Intermediate Compounds 3a and 3b

Chrysin (1 g, 3.93 mmol) was dissolved in DMF (20 mL), and then DIPEA (1.38 mL, 7.86 mmol, 2 equiv.) was added to the mixture. Chloromethyl methyl ether (0.6 mL, 7.86 mmol, 2 equiv.) was then slowly added dropwise to the mixture. The mixture containing all reactants was stirred at room temperature for 12 hours. After the reaction was completed, the resulting mixture was extracted with EtOAc (100 mL×2) and saturated aqueous NH₄Cl solution (100 mL). The organic layer was dried over MgSO₄, filtered under reduced pressure using a filter, and then concentrated. The concentrated product was purified by MPLC (EtOAc/Hexane) to obtain compound 2 (948 mg, 81%).

Compound 2 (413 mg, 1.38 mmol) was dissolved in DMF (20 mL), and then K₂CO₃ (691 mg, 5 mmol, 3.6 equiv.) was added. Allyl bromide (270 μL, 3 mmol, 2.2 equiv.) was then slowly added to the mixture. The mixture was stirred at room temperature for 12 hours, and after the reaction was completed, the resulting mixture was extracted with EtOAc (100 mL×2) and DI water (100 mL). The organic layer was dried over MgSO₄, filtered, and concentrated. The concentrated product was purified by MPLC (EtOAc/Hexane) to obtain compound 3a (524 mg, 93%).

Meanwhile, for compound 3b, compound 2 (400 mg, 1.34 mmol) was dissolved in DMF (20 mL), and K₂CO₃ (278 mg, 2 mmol, 3.6 equiv.) was added. Then, 3,3-dimethylallyl bromide (464 μL, 4 mmol, 3 equiv.) was slowly added to the mixture. The reaction mixture was stirred at room temperature for 12 hours. After completion of the reaction, the resulting mixture was extracted with EtOAc (100 mL×2) and DIW (100 mL). The organic layer was dried over MgSO₄, filtered, and concentrated. The concentrated product was purified by MPLC (EtOAc/Hexane) to obtain compound 3b (371 mg, 76%).

Scheme 1 of the synthesis of intermediate compounds 3a and 3b is as follows:

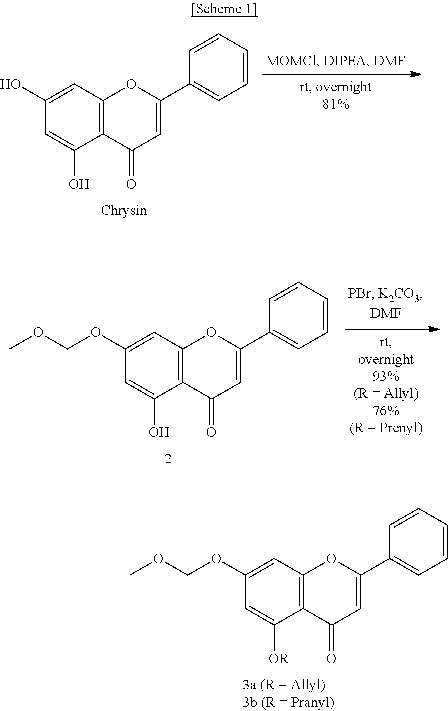

The 7-hydroxy group of chrysin was selectively protected using methoxymethyl (MOM) chloride and DIPEA at room temperature. Then, an allyl or prenyl group was introduced to the 5-hydroxy group of compound 2, generating intermediates 3a and 3b, respectively.

### 2) Synthesis of Compound 5

Compound 3a (414 mg, 1.22 mmol) was dissolved in N,N-dimethylaniline (15 mL) and then stirred at 200° C. for 2 hours. After the reaction was complete, the resulting mixture was cooled to room temperature and extracted with EtOAc (100 mL×3) and 1 M HCl (100 mL). The organic layer was dried over MgSO₄, filtered, and concentrated. The concentrated product was purified by MPLC (EtOAc/Hexane) to obtain compound 4 (358 mg, 87%).

The Claisen rearrangement of 3a was performed at 200° C. using N,N-diethylaniline as a solvent, producing chrysin 5-allyl ether (4) in 87% yield.

Compound 4 (300 mg, 0.82 mmol) was dissolved in methanol (7 mL), and 1 M HCl (3.28 mL, 3.28 mmol, 4 equiv.) solution was added dropwise. The mixture was stirred at 75° C. for 2 hours. After the reaction was complete, the mixture was cooled to room temperature and then extracted with EtOAc (50 mL×2) and water (50 mL). The organic layer was dried over MgSO₄, filtered, and concentrated. The concentrated product was purified by MPLC (EtOAc/Hexane) to obtain compound 5 (240 mg, 82%).

Scheme 2 of the synthesis of compound 5 is as follows:

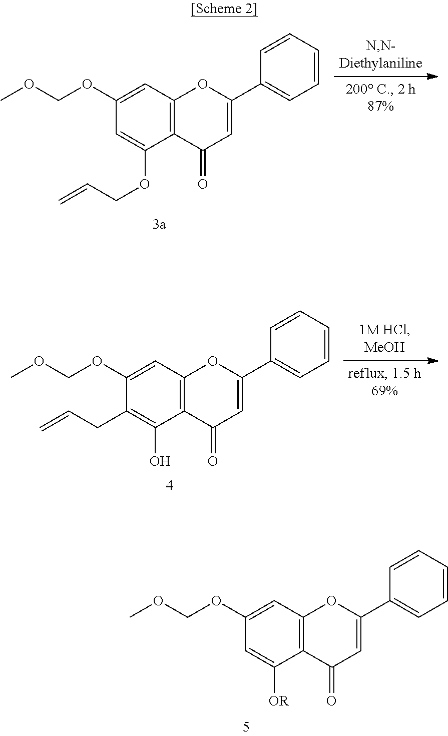

The characterization data for the compound of Formula 5 are as follows: Compound 5: 6-Allyl-5,7-dihydroxy-2-phenyl-4H-chromen-4-one (5). ¹H NMR (700 MHz, CDCl₃): δ 13.10 (s, 1H), 10.96 (s, 1H), 8.08-8.06 (m, 2H), 7.63-7.57 (m, 3H), 6.97 (s, 1H), 6.60 (s, 1H), 4.97 (dq, J=17.10, 2.07 Hz, 1H), 4.93 (dq, J=9.92, 2.10 Hz, 1H), 3.29 (dt, J=6.20 Hz, 1.40, 2H). ¹³C NMR (175 MHz, DMSO-d₆): δ 181.9, 162.9, 162.2, 158.6, 155.4, 135.7, 131.9, 130.8, 129.1, 126.3, 114.6, 109.3, 105.1, 103.7, 93.3, 26.0. HR-MS (EI): calcd for C₁₈H₁₄O₄[M]+ 294.0892; found, 294.0890.

### 3) Synthesis of Compounds 6 and 7

Compound 3b (400 mg, 1.09 mmol) was dissolved in N,N-diethylaniline (15 mL) and then stirred at 230° C. for 3 hours. After the reaction was complete, the resulting mixture was cooled to room temperature and extracted with EtOAc (100 mL×3) and 1 M HCl (100 mL). The organic layer was dried over MgSO₄, filtered, and concentrated. The concentrated product was purified by MPLC (EtOAc/Hexane) to yield compound 6 (112 mg, 28%) and compound 7 (228 mg, 57%).

Scheme 3 of Synthesis of Compounds 6 and 7 is as follows:

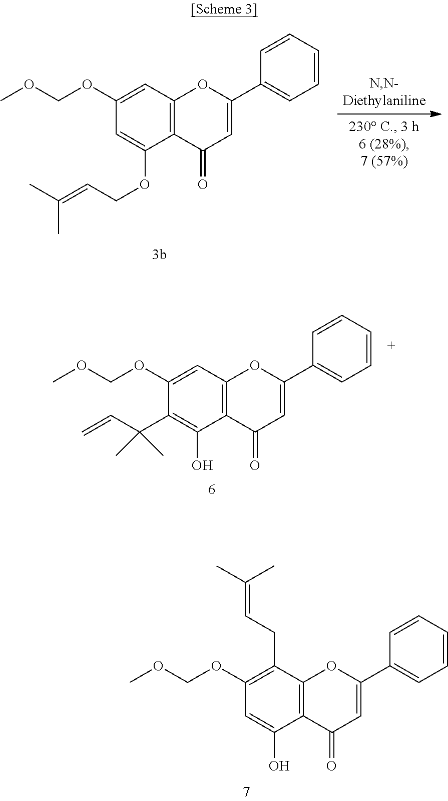 [SMILES: COCOc1cc(OCC=C(C)C)c2c(=O)cc(-c3ccccc3)oc2c1 ; C=CC(C)(C)c1c(OCOC)cc2oc(-c3ccccc3)cc(=O)c2c1O ; COCOc1cc(O)c2c(=O)cc(-c3ccccc3)oc2c1CC=C(C)C]

### 4) Synthesis of Compound 8

Compound 7 (91 mg, 0.25 mmol) was dissolved in methanol (4 mL), and 2 M HCl (500 μL, 1.0 mmol, 4 equiv.) was added dropwise. The mixture was stirred at 75° C. for 2 hours. Upon completion of the reaction, the mixture was cooled to room temperature and extracted with EtOAc (50 mL×2) and water (50 mL). The organic layer was dried over MgSO₄, filtered, and concentrated. The concentrated product was purified by MPLC (EtOAc/Hexane) to yield compound 8 (57 mg, 72%).

Scheme 4 of synthesis of compound 8 is as follows:

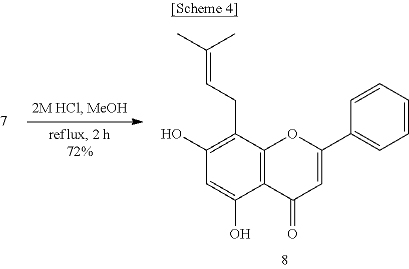 [SMILES: CC(C)=CCc1c(O)cc(O)c2c(=O)cc(-c3ccccc3)oc12]

The characterization data for the compound of Formula 8 are as follows: Compound 8: 5,7-Dihydroxy-8-(3-methylbut-2-en-1-yl)-2-phenyl-4H-chromen-4-one. 1H NMR (700 MHz, DMSO-d6): δ 12.76 (s, 1H), 10.84 (s, 1H), 8.05-8.03 (m, 2H), 7.61-7.57 (m, 3H), 6.95 (s, 1H), 6.31 (s, 1H), 5.21-5.19 (m, 1H), 3.45 (d, J=6.84 Hz, 1H), 1.76 (s, 1H), 1.63 (s, 1H). 13C NMR (175 MHz, DMSO-d6): δ 182.1, 163.0, 161.9, 159.1, 154.6, 131.9, 131.04, 131.0, 129.1, 126.2, 122.4, 106.2, 104.9, 103.9, 98.5, 25.4, 21.3, 17.8. HR-MS (EI): calcd for C20H18O4 [M]+ 322.1205; found, 322.1207.

### 5) Synthesis of Compound 9

Chrysin (254 mg, 1 mmol) was dissolved in DMF (20 mL), and then, K₂CO₃ (552 mg, 4 mmol, 4 equiv.) was added. Allyl bromide (270 μL, 3 mmol, 3 equiv.) was then added dropwise to the mixture. The reaction mixture was stirred at room temperature for 18 hours and extracted with EtOAc (100 mL×2) and water (100 mL). The organic layer was dried over MgSO₄, filtered, and concentrated. The concentrated product was purified by MPLC (EtOAc/Hexane) to yield compound 9 (282 mg, 84%).

### 6) Synthesis of Compound 10

Compound 9 (708 mg, 2.12 mmol) was dissolved in N,N-diethylaniline (20 mL), and then, the mixture was stirred at 200° C. for 2 hours. After the reaction was complete, the resulting mixture was cooled to room temperature and extracted with EtOAc (100 mL×3) and 1 M HCl (100 mL). The organic layer was dried over MgSO₄, filtered, and concentrated. The concentrated product was purified by MPLC (EtOAc/Hexane) to yield compound 10 (527 mg, 74%).

Scheme 5 of the synthesis of compound 10 is as follows:

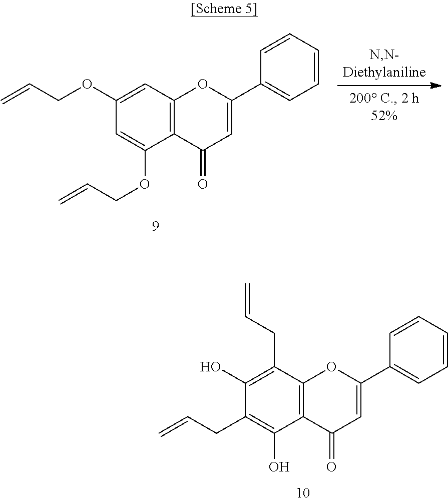 [SMILES: C=CCOc1cc(OCC=C)c2c(=O)cc(-c3ccccc3)oc2c1 ; C=CCc1c(O)c(CC=C)c2oc(-c3ccccc3)cc(=O)c2c1O]

### 7) Synthesis of Compound 1a

Compound 4 (240 mg, 0.7 mmol) was dissolved in THF-H₂O solution (10 mL, 9:1), and then, osmium tetroxide (16 mg, 0.07 mmol, 0.1 equiv.) and 4-methylmorpholine N-oxide (165 mg, 1.4 mmol, 2 equiv.) were added. The mixture was stirred at room temperature for 1 hour and saturated aqueous sodium bisulfite was added at 0° C. to complete the reaction. The mixture was extracted with EtOAc (100 mL×2) and water (100 mL). The organic layer was dried over MgSO₄, and filtered, and the solvent was evaporated under reduced pressure.

The crude diol, from which the solvent has been completely evaporated, was dissolved in a methanol—H₂O solution (10 mL, 2:1), and then sodium periodate (180 mg, 0.84 mmol, 1.2 equiv.) was added. After stirring at room temperature for 1 hour, the mixture was extracted with EtOAc (100 mL×2) and water (100 mL). The organic layer was dried over MgSO₄, and filtered, and then, the solvent was evaporated under reduced pressure.

The crude aldehyde, from which the solvent has been completely evaporated, was dissolved in dichloromethane (5 mL). The mixture was then cooled to 0° C. and a solution of sodium borohydride (51 mg, 1.4 mmol, 2 equiv.) dissolved in methanol was added. The reaction was terminated by stirring at room temperature for 30 minutes and adding distilled water (50 mL). The resulting mixture was extracted with EtOAc (50 mL×2), and the organic layer was dried over MgSO₄. The mixture was filtered, and then the solvent was evaporated, and the concentrated product was purified by MPLC (EtOAc/Hexane) to obtain compound 11 (84 mg, 35% over 3 steps).

Compound 11 (60 mg, 0.18 mmol) was dissolved in methanol (4 mL), and then, 1 M HCl (720 μL, 0.72 mmol, 4 equiv.) was slowly added. The mixture was stirred at 75° C. for 2 hours. After the reaction was complete, the resulting mixture was cooled to room temperature and extracted with EtOAc (50 mL×2) and distilled water (50 mL). The organic layer was dried over MgSO₄, filtered, and concentrated. The concentrated product was purified by MPLC (EtOAc/Hexane) to obtain compound 1a (34 mg, 63%).

Scheme 6 of the synthesis of compound 1a is as follows:

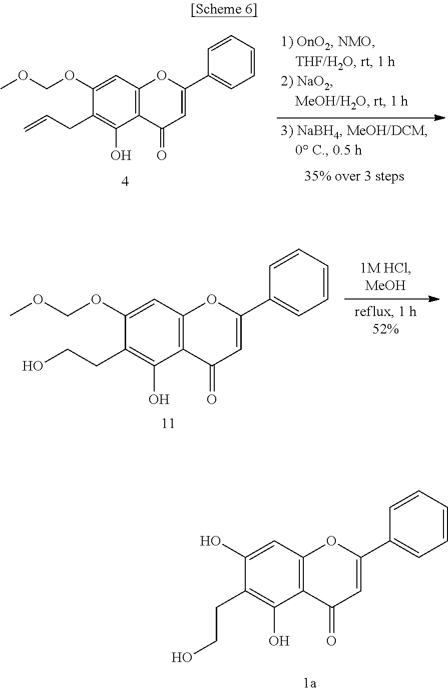 [SMILES: C=CCc1c(OCOC)cc2oc(-c3ccccc3)cc(=O)c2c1O ; COCOc1cc2oc(-c3ccccc3)cc(=O)c2c(O)c1CCO ; O=c1cc(-c2ccccc2)oc2cc(O)c(CCO)c(O)c12]

The characterization data for the compound of Formula 1a are as follows: Compound 1a: 5,7-Dihydroxy-6-(2-hydroxyethyl)-2-phenyl-4H-chromen-4-one (1a). 1H NMR (700 MHz, DMSO-d6): δ 13.11 (s, 1H), 10.92 (s, 1H), 8.08-8.06 (m, 2H), 7.63-7.56 (m, 3H), 6.93 (s, 1H), 6.56 (s, 1H), 4.69 (s, 1H), 3.48 (t, J=7.80 Hz, 2H), 2.76 (t, J=7.56 Hz, 2H). 13C NMR (175 MHz, DMSO-d6): δ 181.9, 162.9, 162.7, 159.0, 155.4, 131.9, 130.8, 129.1, 126.3, 108.5, 105.1, 103.7, 93.4, 59.4, 26.1. HR-MS (EI): calcd for C17H14O5 [M]+ 298.0841; found, 298.0843.

### 8) Synthesis of Compound 1b

Compound 7 (216 mg, 0.6 mmol) was dissolved in a THF—H₂O solution (10 mL, 9:1), and then, osmium tetroxide (14 mg, 0.06 mmol, 0.1 equiv.) and 4-methylmorpholine N-oxide (150 mg, 1.2 mmol, 2 equiv.) were added. The mixture was stirred at room temperature for 1 hour, and the reaction was terminated by the addition of saturated aqueous sodium bisulfite at 0° C. The mixture was extracted with EtOAc (100 mL×2) and water (100 mL). The organic layer was dried over MgSO₄, and filtered, and the solvent was evaporated under reduced pressure.

The crude diol from which the solvent has evaporated was dissolved in a methanol—H₂O solution (10 mL, 2:1), and then, sodium periodate (154 mg, 0.72 mmol, 1.2 equiv.) was added. After stirring at room temperature for 1 hour, the mixture was extracted with EtOAc (100 mL×2) and water (100 mL). The organic layer was dried over MgSO₄, and filtered, and the solvent was evaporated under reduced pressure.

The crude aldehyde, from which the solvent has been completely evaporated, was dissolved in dichloromethane (5 mL). The mixture was then cooled to 0° C. and a solution of sodium borohydride (45 mg, 1.2 mmol, 2 equiv.) dissolved in methanol was added. The mixture was stirred at room temperature for 30 minutes and distilled water (50 mL) was added to terminate the reaction. The mixture was extracted with EtOAc (50 mL×2), and the organic layer was dried over MgSO₄. The mixture was filtered, and then the solvent evaporated, and the concentrated product was purified by MPLC (EtOAc/Hexane) to obtain compound 12 (78 mg, 38% over 3 steps).

Compound 12 (78 mg, 0.23 mmol) was dissolved in methanol (4 mL), and then, 2 M HCl (460 μL, 0.92 mmol, 4 equiv.) was slowly added. The mixture was stirred at 75° C. for 12 hours. After the reaction was complete, the resulting mixture was cooled to room temperature and extracted with EtOAc (50 mL×2) and distilled water (50 mL). The organic layer was dried over MgSO₄, filtered, and concentrated. The concentrated product was purified by MPLC (EtOAc/Hexane) to yield compound 1b (48 mg, 70%).

Scheme 7 of the synthesis of compound 1b is as follows:

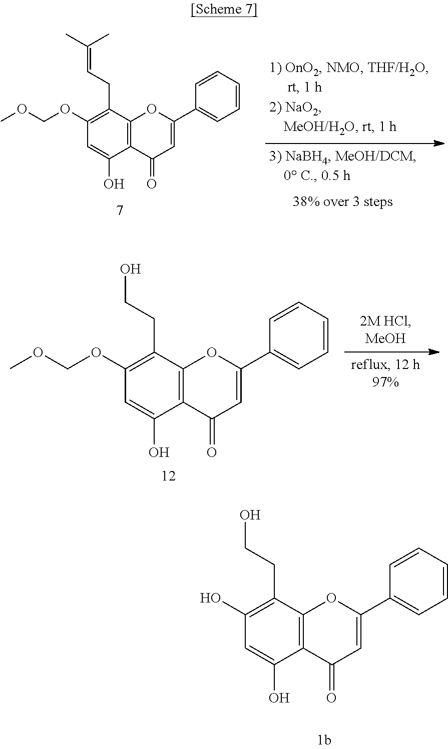 [SMILES: COCOc1cc(O)c2c(=O)cc(-c3ccccc3)oc2c1CC=C(C)C ; COCOc1cc(O)c2c(=O)cc(-c3ccccc3)oc2c1CCO ; O=c1cc(-c2ccccc2)oc2c(CCO)c(O)cc(O)c12]

The characterization data for the compound of Formula 1b are as follows: Compound 1b: 5,7-Dihydroxy-8-(2-hydroxyethyl)-2-phenyl-4H-chromen-4-one (1b). 1H NMR (500 MHz, DMSO-d6): δ 12.81 (s, 1H), 10.80 (bs, 1H), 8.10-8.09 (m, 2H), 7.63-7.59 (m, 3H), 6.97 (s, 1H), 6.31 (s, 1H), 4.82 (bs, 1H), 3.57 (t, J=7.51 Hz, 2H), 2.95 (t, J=7.39 Hz, 2H). 13C NMR (125 MHz, DMSO-d6): δ 182.3, 163.1, 162.4, 159.3, 155.2, 132.0, 131.1, 129.2, 126.4, 104.8, 103.9(103.91), 103.9(103.89), 98.5, 60.0, 26.5. HR-MS (EI): calcd for C17H14O5 [M]+ 298.0841; found, 298.0843.

### 2. RAW264.7 Cell Culture and Cell Viability Measurement for Anti-Inflammatory Activity Evaluation

Mouse macrophage cell lines (RAW264.7) were cultured in high-glucose DMEM (Dulbecco's modified Eagle's medium; WelGene) medium supplemented with 10% fetal bovine serum (Gibco BRL) and 100 U/mL penicillin/streptomycin (Invitrogen) at 37° C. in an incubator containing 5% CO₂. To evaluate cytotoxicity, WST-1 assay was performed using the EZ-Cytox cell viability assay kit (DAEIL Lab). RAW264.7 cells were seeded in 96-well plates at 5×103 cells/well and then cultured for 4 hours, and were respectively treated with each of chrysin derivatives 1a, 1b, 5, 8, and 10, and chrysin (Chry) dissolved in 0.1% dimethyl sulfoxide (DMSO)/DMEM, at respective concentrations (2.5, 5, and 10 μM), with or without LPS (200 ng/mL). “LPS only” refers to treatment with LPS and 0.1% DMSO. The DMEM treatment group containing 0.1% DMSO served as the control group. After 18 hours of culture, cell viability was measured using the Ez-cytox reagent.

In the experiments and drawings below, “Control” represents the DMEM treatment group with 0.1% DMSO, “Chry” represents chrysin, and “LPS only” represents LPS and 0.1% DMSO treatment. 1a, 1b, 5, 8, and 10 represent chrysin derivatives of respective chemical formulas thereof, respectively.

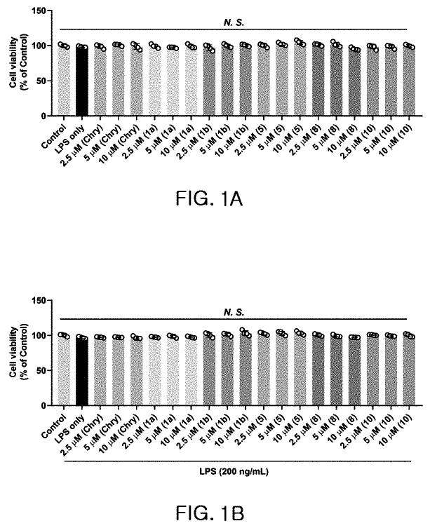

To determine the optimal concentration for evaluating the efficacy of inflammatory response control, the cytotoxicity of chrysin (Chry) and chrysin derivatives (compounds 1a, 1b, 5, 8, and 10) was evaluated using the WST-1 assay in LPS (200 ng/mL)-treated and -untreated conditions. As shown in FIG. 1A, all tested compounds showed no significant cytotoxicity in RAW264.7 cells when treated with 2.5, 5, and 10 μM concentrations for 18 hours. Additionally, the presence of LPS (200 ng/mL) did not affect cell viability under experimental conditions (FIG. 1B). Based on these results, the above concentration was selected for subsequent anti-inflammatory effect evaluation.

### 3. Measurement of IL-6, TNF-α and PGE-2, and COX-2 Protein Expression for Anti-Inflammatory Activity

### 1) Evaluation of IL-6 Inhibitory Activity of Chrysin Derivatives

The levels of IL-6, TNF-α, and PGE-2, key mediators of the inflammatory response, in cell culture supernatants were analyzed using enzyme-linked immunosorbent assay. RAW264.7 cells were seeded at 0.5×105 cells/well in 48-well plates and then cultured for 4 hours. After incubation, cells were treated with synthetic samples dissolved in 0.1% DMSO/DMEM at concentrations of 2.5, 5, and 10 μM, respectively, along with LPS (200 ng/mL) for 18 hours. After 18 hours of incubation, the culture medium was collected, and the levels of IL-6, TNF-α, and PGE-2 were measured. IL-6 and TNF-α levels were measured using a mouse CBA ELISA kit (BD Biosciences), and PGE-2 levels were measured using a mouse ELISA kit (CUSABIO).

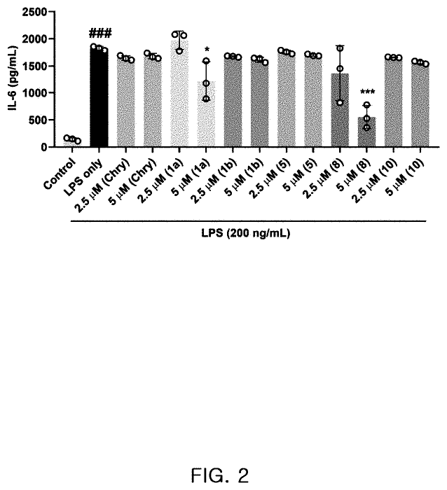

IL-6 is primarily produced by macrophages in response to infection and tissue damage, and elevated IL-6 levels are associated with chronic inflammation, making it an important target for anti-inflammatory therapies. In this study, the potential of the synthesized chrysin derivatives to inhibit IL-6 production in LPS-stimulated RAW264.7 cells was evaluated. Among the tested compounds, 1a and 8 significantly inhibited the level of IL-6 increased by LPS when treated at a concentration of 5 μM. Other derivatives 1b, 5, and 10 including chrysin did not exhibit significant IL-6 inhibitory effects (FIG. 2).

### 2) Evaluation of COX-2 Inhibitory Activity and TNF-α and PGE-2 Measurement of Chrysin Derivatives

To verify the anti-inflammatory effects of compounds 1a and 8, which exhibited IL-6 inhibitory activity, In comparison with the starting material, chrysin, a reevaluation of IL-6 and an evaluation of inhibitory activity on other major inflammatory mediators, TNF-α, PGE2, and COX-2, were conducted. TNF-α is a key cytokine mediating the early inflammatory response and is known to be elevated in various inflammatory and autoimmune diseases.

COX-2, induced by inflammatory stimuli, promotes the synthesis of various prostaglandins, including PGE2, and amplifies the inflammatory response at the site of inflammation through pain, fever, increased vascular permeability, or the like. Therefore, COX-2 and PGE2 are emerging as key targets for anti-inflammatory drug development.

Intracellular COX-2 expression levels were measured using Western blotting. After harvesting, cells were lysed with 250 μL of radioimmunoprecipitation assay buffer containing protein degradation inhibitors and kept on ice for 15 minutes. The cell lysate was then collected and centrifuged at 16,000×g for 20 minutes at 4° C., and the protein concentration of the sample was measured using a BCA protein assay kit. 15 μL of the lysed sample was separated by 10% sodium dodecyl sulfate-polyacrylamide gel electrophoresis (SDS-PAGE) and transferred to a polyvinylidene difluoride membrane (PVDF). The membrane was blocked for 1 hour, incubated overnight at 4° C. with primary antibodies (anti-COX-2 and β-actin), and then incubated with horseradish peroxidase (HRP)-conjugated secondary antibodies for 1 hour at room temperature. The membrane was visualized using ECL Western blotting detection reagents using a ChemiDoc Imaging System.

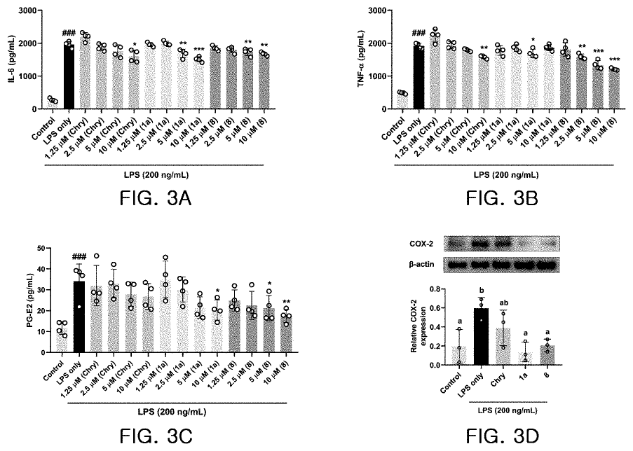

According to FIG. 3A, chrysin exhibited an IL-6 inhibitory effect at 10 μM, while compounds 1a and 8 exhibited significant IL-6 inhibitory effects at 5 and 10 μM. Similarly, FIG. 3B showed that chrysin exhibited a TNF-α inhibitory effect only at 10 μM, while compounds 1a and 8 exhibited significant TNF-α inhibitory effects at 5 and 10 μM.

According to FIG. 3C, the results of measuring the PGE2 inhibitory activity of chrysin and compounds 1a and 8 showed that chrysin did not show significant PGE2 inhibitory activity at all concentrations, but compound 1a inhibited PGE2 at a concentration of 10 μM, and compound 8 inhibited PGE2 in a concentration-dependent manner at concentrations of 5 μM and 10 μM. Furthermore, chrysin did not significantly inhibit COX-2 expression compared to the LPS group, whereas compounds 1a and 8 inhibited COX-2 expression compared to the LPS group (FIG. 3D).

In conclusion, it can be concluded that compounds 1a and 8 exhibit superior anti-inflammatory effects induced by LPS stimulation compared to the starting material, chrysin.

### 4. Evaluation of Modulation Potential of Immune Response Involved in Granuloma Formation

Pulmonary granulomas, a key pathological feature in tuberculosis treatment, serve as a crucial barrier to drug penetration. Host-directed therapy (HDT), targeting these granulomas, has recently been proposed as a strategy to improve treatment efficacy and more effectively manage infection. Therefore, the inhibitory effects of inflammatory mediators TNF-α and IL-6 in relation to pulmonary granulomas formation were evaluated. TNF-α plays an essential role in the formation and maintenance of pulmonary granulomas and contributes to the isolation and elimination of Mycobacterium tuberculosis by recruiting and activating immune cells. IL-6 amplifies the inflammatory response and activates immune cells, thereby enhancing the immune response against Mycobacterium tuberculosis. However, this amplification of the inflammatory response leads to tissue damage and functional decline. In particular, the formation of pulmonary granulomas may cause Mycobacterium tuberculosis to enter a dormant state, contributing to resistance and recurrent tuberculosis.

To evaluate the mediators associated with pulmonary granuloma formation induced by Mycobacterium tuberculosis infection, trehalose-6,6′-dimycolate (TDM), derived from Mycobacterium bovis tuberculosis, was dissolved in hexane and ethanol, and coated on a 96-well plate at 1 μg/well, and then, the solvent was evaporated in a clean bench. RAW264.7 cells were seeded, treated with chrysin and its derivatives after 2 hours, and cultured for 18 hours. TNF-α and IL-6 levels in the culture supernatant were analyzed using an ELISA kit.

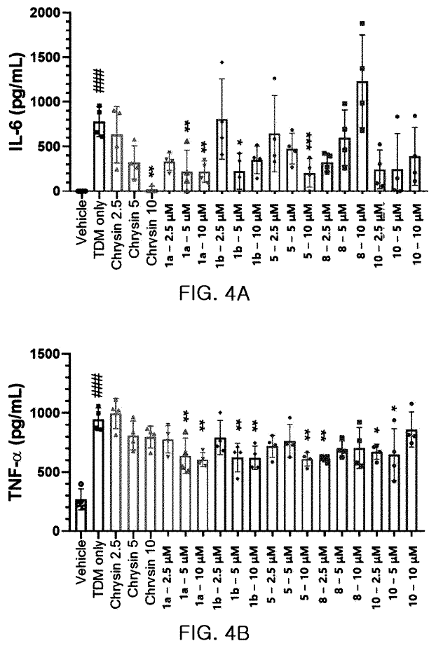

According to FIG. 4A, the IL-6 level increased by TDM stimulation, a glycolipid derived from Mycobacterium tuberculosis, was significantly suppressed by 10 μM chrysin. Compound 1a inhibited IL-6 at concentrations of 5 μM and 10 μM, while compound 1b inhibited IL-6 at 5 μM, but without concentration dependence. Compound 5 inhibited IL-6 at 10 μM.

According to FIG. 4B, for TNF-α, chrysin did not show a significant inhibitory effect at all concentrations compared to the TDM-stimulated group. Compounds 1a and 1b inhibited TNF-α at concentrations of 5 μM and 10 μM. Compound 5 inhibited TNF-α at 10 μM, compound 8 at concentration of 2.5 μM, and compound 10 at concentrations of 2.5 μM and 5 μM.

In FIGS. 4A and 4B, “Vehicle” refers to the solvent-injected group without TDM, and “TDM only” refers to the group treated with TDM alone.

In conclusion, the inhibitory effect of inflammatory mediators by TDM derived from Mycobacterium tuberculosis was superior to that of chrysin in that compounds 1a, 1b, and 5 significantly inhibited both TNF-α and IL-6.

As set forth above, a novel chrysin derivative provided in an embodiment of the present disclosure exhibits excellent anti-inflammatory activity, and has an excellent effect of suppressing inflammatory mediators by glycolipids derived from Mycobacterium tuberculosis, and may further regulate immune responses involved in granuloma formation, and is therefore expected to be effectively applied to the treatment of tuberculosis-related inflammation and tuberculosis.

While example embodiments have been shown and described above, it will be apparent to those skilled in the art that modifications and variations could be made without departing from the scope of the present disclosure as defined by the appended claims.

What is claimed is:

1. A chrysin derivative compound or a salt thereof, the chrysin derivative compound being represented by chemical formula 1, 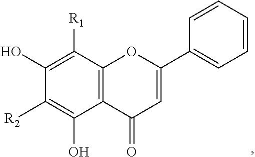
wherein in chemical formula 1, at least one of R₁ and R₂ is hydrogen, and the other is a C₁₋₆ hydroxyalkyl group or a C₁₋₁₀ alkenyl group unsubstituted or substituted with one or more C₁₋₁₀ alkyl groups.

2. The chrysin derivative compound or the salt thereof of claim 1, wherein in chemical formula 1, at least one of R₁ and R₂ is hydrogen, and the other is hydroxyethyl, or a C₁₋₁₀ allyl group unsubstituted or substituted with one or two methyl groups.

3. The chrysin derivative compound or the salt thereof of claim 1, wherein the chrysin derivative compound represented by chemical formula 1 is at least one selected from the group consisting of Compound 1a, Compound 1b, Compound 5, and Compound 8:

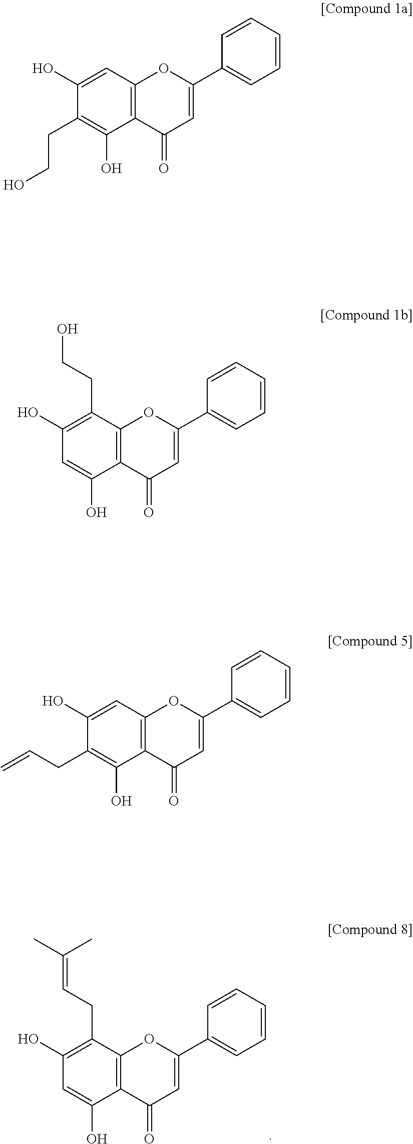 [SMILES: O=c1cc(-c2ccccc2)oc2cc(O)c(CCO)c(O)c12 ; O=c1cc(-c2ccccc2)oc2c(CCO)c(O)cc(O)c12 ; C=CCc1c(O)cc2oc(-c3ccccc3)cc(=O)c2c1O ; CC(C)=CCc1c(O)cc(O)c2c(=O)cc(-c3ccccc3)oc12]

4. A composition for a treatment of tuberculosis, comprising the chrysin derivative compound represented by chemical formula 1 of claim 1 or a salt thereof as an active ingredient.

5. The composition for a treatment of tuberculosis of claim 4, wherein the tuberculosis is derived from a strain of genus Mycobacterium.

6. The composition for a treatment of tuberculosis of claim 5, wherein the strain of genus Mycobacterium is one or more strains selected from the group consisting of Mycobacterium tuberculosis erdman, Mycobacterium tuberculosis, Mycobacterium bovis, Mycobacterium africanum, Mycobacterium microti, Mycobacterium fortuitum, Mycobacterium avium, Mycobacterium xenopi, Mycobacterium smegmatis, Mycobacterium tuberculosis H37Rv, Mycobacterium tuberculosis K, Mycobacterium kansasii, Mycobacterium marinum, Mycobacterium fortuitum, and Mycobacterium chelonae.

7. The composition for a treatment of tuberculosis of claim 4, wherein the tuberculosis is at least one of extrapulmonary tuberculosis and pulmonary tuberculosis developed in an area of at least one of the lymph nodes, gastrointestinal tract, joints, meninges, and genitourinary organs.

8. A composition for a treatment of inflammation, comprising the chrysin derivative compound represented by chemical formula 1 of claim 1 or a salt thereof as an active ingredient.

9. The composition for a treatment of inflammation of claim 8, wherein the inflammation is inflammation caused by tuberculosis.

10. The composition for a treatment of inflammation of claim 9, wherein the inflammation caused by the tuberculosis is at least one selected from the group consisting of chronic inflammation and granuloma.

11. The composition for a treatment of inflammation of claim 8, wherein the chrysin derivative compound represented by chemical formula 1 or the salt thereof inhibits an inflammatory mediator caused by Trehalose-6,6′-dimycolate (TDM).

12. The composition for a treatment of inflammation of claim 11, wherein the inflammatory mediator is at least one of TNF-α and IL-6.

13. A method for treating tuberculosis, comprising administering to a subject in need thereof the chrysin derivative compound represented by chemical formula 1 of claim 1 or a salt thereof as an active ingredient.

14. The method of claim 13, wherein the tuberculosis is derived from a strain of genus Mycobacterium.

15. The method of claim 14, wherein the strain of genus Mycobacterium is one or more strains selected from the group consisting of Mycobacterium tuberculosis erdman, Mycobacterium tuberculosis, Mycobacterium bovis, Mycobacterium africanum, Mycobacterium microti, Mycobacterium fortuitum, Mycobacterium avium, Mycobacterium xenopi, Mycobacterium smegmatis, Mycobacterium tuberculosis H37Rv, Mycobacterium tuberculosis K, Mycobacterium kansasii, Mycobacterium marinum, Mycobacterium fortuitum, and Mycobacterium chelonae.

16. The method of claim 13, wherein the tuberculosis is at least one of extrapulmonary tuberculosis and pulmonary tuberculosis developed in an area of at least one of the lymph nodes, gastrointestinal tract, joints, meninges, and genitourinary organs.

17. A method for treating inflammation, comprising administering to a subject in need thereof the chrysin derivative compound represented by chemical formula 1 of claim 1 or a salt thereof as an active ingredient.

18. The method of claim 17, wherein the inflammation is inflammation caused by tuberculosis or is at least one selected from the group consisting of chronic inflammation and granuloma.

19. The method of claim 17, wherein the chrysin derivative compound represented by chemical formula 1 or the salt thereof inhibits an inflammatory mediator caused by Trehalose-6,6′-dimycolate (TDM).

20. The method of claim 19, wherein the inflammatory mediator is at least one of TNF-α and IL-6.
